SEQUENCE LISTING (10 sequences)

SEQ ID NO: 1 - DNA, 663 bp, Richadella dulcifica
  CDS (1)..(663)
atgaaggaattaacaatgctctctctctcgttcttcttcgtctctgcattgttggcagcagcggccaacccactgcttagtgcagcggattcggcacccaatccggttcttgacatagacggagagaaactccggacggggaccaattattacattgtgccggtgctccgcgaccatggcggcggccttacagtatccgccaccacccccaacggcaccttcgtttgtccacccagagttgtccaaacacgaaaggaggtcgaccacgatcgccccctcgctttctttccagagaacccaaaggaagacgttgttcgagtctccaccgatctcaacatcaatttctcggcgttcatgccctgtcgttggaccagttccaccgtgtggcggctcgacaaatacgatgaatccacggggcagtacttcgtgaccatcggcggtgtcaaaggaaacccaggtcccgaaaccattagtagctggtttaagattgaggagttttgtggtagtggtttttacaagcttgttttctgtcccaccgtttgtggttcctgcaaagtaaaatgcggagatgtgggcatttacattgatcagaagggaagaaggcgtttggctctcagcgataaaccattcgcattcgagttcaacaaaaccgtatacttctaa

SEQ ID NO: 2 - protein, 220 aa, Richadella dulcifica
MKELTMLSLSFFFVSALLAAAANPLLSAADSAPNPVLDIDGEKLRTGTNYYIVPVLRDHGGGLTVSATTPNGTFVCPPRVVQTRKEVDHDRPLAFFPENPKEDVVRVSTDLNINFSAFMPCRWTSSTVWRLDKYDESTGQYFVTIGGVKGNPGPETISSWFKIEEFCGSGFYKLVFCPTVCGSCKVKCGDVGIYIDQKGRRRLALSDKPFAFEFNKTVYF

SEQ ID NO: 3 - DNA, 29 bp, Artificial Sequence
  Synthetic primer
ttttctagaatgaaggaattaacaatgct

SEQ ID NO: 4 - DNA, 29 bp, Artificial Sequence
  Synthetic primer
tttgagctcttagaagtatacggttttgt

SEQ ID NO: 5 - DNA, 29 bp, Artificial Sequence
  Synthetic primer
taggatccgattcggcacccaatccggtt

SEQ ID NO: 6 - DNA, 29 bp, Artificial Sequence
  Synthetic primer
ttctcgaggaacgccgagaaattgatgtt

SEQ ID NO: 7 - DNA, 69 bp, Artificial Sequence
  Synthetic primer
attacattttacattctacaactacatctagaggccaaatcggccatgaaggaattaacaatgctctct

SEQ ID NO: 8 - DNA, 70 bp, Artificial Sequence
  Synthetic primer
cgagctcgcggccgccccggggatcctctagaggcccttatggccttagaagtatacggttttgttgaac

SEQ ID NO: 9 - DNA, 68 bp, Artificial Sequence
  Synthetic primer
attacattttacattctacaactacatctagaggccaaatcggccatggattcggcacccaatccggt

SEQ ID NO: 10 - DNA, 759 bp, Synsepalum dulcificum
acaacaatgaaggaattaacaatgctctctctctcgttcttcttcgtctctgcattgttggcagcagcggccaacccactgcttagtgcagcggattcggcacccaatccggttcttgacatagacggagagaaactccggacggggaccaattattacattgtgccggtgctccgcgaccatggcggcggccttacagtatccgccaccacccccaacggcaccttcgtttgtccacccagagttgtccaaacacgaaaggaggtcgaccacgatcgccccctcgctttctttccagagaacccaaaggaagacgttgttcgagtctccaccgatctcaacatcaatttctcggcgttcatgccctgtcgttggaccagttccaccgtgtggcggctcgacaaatacgatgaatccacggggcagtacttcgtgaccatcggcggtgtcaaaggaaacccaggtcccgaaaccattagtagctggtttaagattgaggagttttgtggtagtggtttttacaagcttgttttctgtcccaccgtttgtggttcctgcaaagtaaaatgcggagatgtgggcatttacattgatcagaagggaagaaggcgtttggctctcagcgataaaccattcgcattcgagttcaacaaaaccgtatacttctaattgggtttgggggtggtttttccaatcacatctcatgtatgatcagctccattatcgatctgcataattataattaataaggaagctttt

(19) United States
(12) Patent Application Publication
(10) Pub. No.: US 2009/0205068 A1
(43) Pub. Date: Aug. 13, 2009

(54) Method for Producing Genetically Modified Plant Expressing Miraculin

(75) Inventors: Hiroshi Ezura, Ibaraki (JP); Hyeon-Jin Sun, Ibaraki (JP); Masanobu Kirita, Ibaraki (JP); Takanari Ichikawa, Kanagawa (JP); Nobuyo Nishizaki, Kanagawa (JP)

Correspondence Address: BIRCH STEWART KOLASCH & BIRCH, PO BOX 747, FALLS CHURCH, VA, 22040-0747, US

(73) Assignees: Hiroshi EZURA, Tsukuba-shi (JP); INPLANTA INNOVATIONS INC, Yokohama-shi (JP)

(21) Appl. No.: 11/659,342

(22) PCT Filed: Aug. 4, 2005
(86) PCT No.: PCT/JP05/14705

§ 371 Date: Jan. 3, 2008

(30) Foreign Application Priority Data
Aug. 6, 2004 (JP) 2004-230205

(51) Int. Cl. A01H 5/00; A61K 38/16

(52) U.S. Cl. 800/278; 800/305; 800/317.4; 514/2

(57) ABSTRACT

The present invention relates to: a method for producing a transgenic plant expressing miraculin, comprising introducing a miraculin gene into a useful plant that can be easily produced and cultivated throughout the year using plant molecular breeding technology; a transgenic plant expressing miraculin, which is produced by the method; and the use thereof. The present invention also relates to a food additive, a food or drink, or an antidiabetic drug, which contains miraculin that is produced by the transgenic plant.

### TECHNICAL FIELD

The present invention relates to the production and the use of a genetically modified plant expressing miraculin.

### BACKGROUND ART

Miraculin is a taste-modifying protein contained in the fruit of a miracle fruit plant (Richadella dulcifica). Miraculin was identified in 1968 to be a functional ingredient of miracle fruit (Kurihara and Beidler, Science (1968) 161 (847), pp. 1241-1243). Thereafter, the complete amino acid sequence of the miraculin protein was determined (Theerasilp et al., J Biol Chem. (1989) 264 (12), pp. 6655-6659) and the nucleotide sequence of the gene was also determined (Masuda et al., Gene (1995) 161, pp. 175-177). The full-length cDNA sequence of the miraculin gene has been reported in Masuda et al., Gene (1995) 161, pp. 175-177. Miraculin has an effect of modifying sourness of a food to sweetness. For example, when a person eats a lemon after eating miracle fruit, he or she experiences a sweet taste like that of an orange. Because of such effect, the fruit is called “miracle fruit.” The miracle fruit plant is a native plant of tropical West Africa. The miracle fruit is traditionally eaten by local people before drinking or eating sour beverages or foods such as palm wine or corn bread.

Employing such function of miraculin, it has been attempted to use miracle fruit as a supplementary food useful for dieting to reduce body weight or to treat diabetes by reducing calorie intake from diets. Eating miracle fruit before a meal can result in reduced sugar intake and a sufficient feeling of fullness by eating foods with a sour taste. Hence, meal size can be comfortably reduced. As a result, a person can diet for controlling body weight or dietary therapy without feeling any mental stress. To use miracle fruit for these applications, stable production and supply of the fruit are required.

However, to date, miracle fruit has not been stably produced or supplied in sufficient amounts. Hence, miracle fruit has not been provided sufficiently for these purposes. Since miracle fruit is a native plant of the tropics, it requires full-year high-temperature cultivation conditions when it is cultivated in non-tropical areas. Accordingly, miracle fruit is cultivated in a greenhouse or the like that is heavily equipped with a heating system, for example. Furthermore, miraculin, the functional ingredient, is easily degraded in the fruits, so that the production of miraculin fruit in tropical areas and the supply of miraculin in a sufficient amount therefrom in non-tropical areas are difficult in terms of technology and cost. Development of technology for stable low-cost and year-round supply of miracle fruit is essential for effectively utilizing the miraculin activity of miracle fruit. Miraculin itself is also expected to be used as a supplementary food useful for dieting for controlling body weight or dietary therapy to treat diabetes, for example. However, it is currently difficult to supply miraculin in a sufficient amount, as described above.

Kurihara et al. have reported that a protein reacting with a miraculin-specific antibody can be produced in yeast that has been caused to express a miraculin gene after the introduction of the gene thereinto, but the protein lacks sweetness-inducing activity (Kurihara et al., Foods & Food Ingredients Journal of Japan No. 174 (1997), “Structures and Activities of Sweetness-Inducing Substances (Miraculin, Curculin, and Strogin) and the Heat-stable Sweet Protein, Mabinlin”). In the review of Kurihara et al., it was also reported that a protein reacting with a miraculin-specific antibody can be produced by expressing the miraculin gene in tobacco cells. However, whether the miraculin gene product expressed in tobacco possesses sweetness-inducing activity has not been reported. Successful production of miraculin having sweetness-inducing activity through the introduction of the miraculin gene into a plant and the expression of the same in the plant has not been known to date.

### DISCLOSURE OF THE INVENTION

The present invention has been achieved in the above-described circumstances. An object of the present invention is to provide a method for producing a useful genetically modified plant that produces miraculin and use of miraculin.

The present inventors have worked on studies concerning the introduction and expression of foreign genes into useful plants that can be cultivated throughout the year in most areas in the world, such as lettuce and tomato, and into useful plants from which fruits can be harvested over a long period of time, such as strawberry.

The present inventors have attempted to introcuce the miraculin gene into useful plants that can be easily produced and cultivated throughout the year using plant molecular breeding technology. As a result, the present inventors have succeeded in the production of a genetically modified plant expressing miraculin.

As a result of analysis using the thus produced recombinant plant using a miraculin-specific antibody, it was found that the recombinant plant expressed miraculin with a concentration equivalent to that in miracle fruit and in a functional form, i.e., dimer.

The thus completed present invention relates to a plant expressing miraculin, a method for producing the same, and use of the same.

One aspect of the present invention is a method for producing a genetically modified plant, comprising introducing a miraculin gene into a plant and expressing miraculin in the plant. Miraculin cDNA can be isolated from miracle fruit and then used as a miraculin gene. Furthermore, when the miraculin gene is inserted into a vector and then the vector is introduced into Agrobacterium, the resulting Agrobacterium can be used for gene transfer into plant cells. In the present invention, miraculin produced by a genetically modified plant that is produced by such method can be used as a food additive or a pharmaceutical preparation, and particularly as an antidiabetic drug.

Another aspect of the present invention is a transgenic plant having taste-modifying activity into which a miraculin gene has been introduced. A preferable miraculin gene is at least one of the following miraculin genes (a) to (f):

(a) a gene comprising the nucleotide sequence as shown in SEQ ID NO: 1;

(b) a gene comprising a DNA that hybridizes under stringent conditions to the DNA comprising the nucleotide sequence as shown in SEQ ID NO: 1 and encodes a protein having taste-modifying activity;

(c) a gene encoding a protein that comprises the amino acid sequence as shown in SEQ ID NO: 2;

(d) a gene encoding a protein that comprises an amino acid sequence derived from the amino acid sequence as shown in SEQ ID NO: 2 by deletion, substitution, or addition of one or several amino acids and has taste-modifying activity;

(e) a gene comprising a nucleotide sequence that has 70% or more identity with the nucleotide sequence as shown in SEQ ID NO: 1 and encoding a protein that has taste-modifying activity; and

(f) a gene encoding a protein that comprises an amino acid sequence having 85% or more identity with the amino acid sequence as shown in SEQ ID NO: 2 and has taste-modifying activity.

Here, “taste-modifying activity” typically refers to an activity that converts sourness to sweetness.

Furthermore, the above plant is preferably lettuce, tomato, strawberry, or Arabidopsis thaliana. Furthermore, in the present invention, the above transgenic plant is particularly preferably a diploid plant.

Another aspect of the present invention is a method for producing miraculin, comprising extracting miraculin from the above transgenic plant.

Still another aspect of the present invention is a food additive comprising miraculin that is produced in the above transgenic plant. Another aspect of the present invention is a food or drink comprising miraculin that is produced in the above transgenic plant. Such food or drink is more preferably used for a patient with diabetes. Another aspect of the present invention is an antidiabetic drug comprising miraculin that is produced in the above transgenic plant.

The useful plant expressing miraculin has various types of industrial utility. One type of utility is the use of miraculin as a food additive used in diet foods. Acid and miraculin are added to a food (which is normally sweetened with the addition of sugar), instead of adding sugar thereto. In this manner, sweetness can be added to a food while keeping the calories in the food at a low level. Another type of utility is the use of miraculin for dietary therapy for patients with diabetes. Dietary restrictions are imposed on patients with diabetes for the purpose of suppressing excessive sugar intake. Such dietary restrictions cause extreme stress during dietary therapy. However, patients with diabetes can comfortably feel fullness through the intake of food or drink containing miraculin during meals, so as to be able to reduce stress during dietary therapy. Therefore, diabetic therapy can be smoothly performed.

The disclosure in the description and the drawings of Japanese Patent Application No. 2004-230205, to which the present application claims priority, is incorporated herein.

### BRIEF DESCRIPTION OF THE DRAWINGS

FIG. 1 shows the structures of miraculin expression vectors for introducing the miraculin gene into plant cells. pBIEL₂ΩMIR represents a high-expression vector and pBI35SMIR represents a general type of vector. RB indicates the right terminal end of T-DNA, NPTII indicates a neomycin phosphotransferase gene, Tnos indicates a nopaline synthase gene terminator, 35SP indicates a CaMV 35S promoter, and MIR indicates the miraculin gene.

FIG. 2 shows photographs showing the results of Southern analysis for examining the incorporation of the miraculin gene into recombinants. Genomic DNAs were extracted from non-recombinants and 40 lines of recombinants and then subjected to Southern analysis. (A) and (B) Images showing the analysis of the prepared membranes with BAS. (C) and (D) X-ray film images showing the detection of the same membranes by autoradiography. WT indicates a non-recombinant. Lanes 1 to 20 of left-side photographs (A) and (C), represent recombinants into which a binary vector pBIEL₂ΩMIR was introduced. Lanes 1 to 20 of right-side photographs (B) and (D) represent recombinants into which pBI35SMIR was introduced.

FIG. 3 shows photographs showing the results of Northern analysis for examining the transcription of the miraculin gene within recombinants. Total RNAs were extracted from non-recombinants and 19 lines of recombinants and then subjected to Northern analysis. WT indicates a non-recombinant. Nine lanes in the left photograph represent recombinants into which pBIEL₂ΩMIR was introduced. Ten lanes in the right photograph represent recombinants into which pBI35SMIR was introduced.

FIG. 4 shows the results of Western analysis for examining the expression of miraculin in recombinants. Total proteins were extracted from non-recombinants and 18 lines of recombinants and then subjected to Western analysis. WT indicates a non-recombinant. Nine lanes in the left photograph represent recombinants into which pBIEL₂ΩMIR was introduced. Nine lanes in the right photograph represent recombinants into which pBI35SMIR was introduced. M indicates a miraculin protein isolated and purified from miraculin fruit.

FIG. 5 is a photograph showing the results of Southern blot analysis for examining the expression of a miraculin gene in recombinant (transgenic) tomato leaves into which the miraculin gene was introduced. “Wild type” indicates a tomato plant into which no miraculin gene was introduced. The other 14 lanes each denote abbreviations of the names of recombinants produced by the introduction of pBI35SMIR.

FIG. 6 is a photograph showing the results of Western blot analysis, in which protein extracts from the leaves of recombinant (transgenic) tomato individuals into which a miraculin gene had been introduced were developed by SDS-PAGE under non-reducing conditions, followed by detection of miraculin. “Marker” indicates a protein molecular weight marker. “Miraculin” indicates a miraculin protein isolated from miraculin fruit. “Wild type” indicates a tomato plant into which no miraculin gene was introduced. “1 copy” lanes 2-1, 5-2, 7C, 11A, 14-3, 21-4, 56B, 58-1, and 64-2 indicate the names of transformants into which 1 copy of the miraculin gene was introduced. “2 copies” lanes 4A, 20-1, and 22A indicate the names of transformants into which 2 copies of the miraculin gene were introduced. “Multiple copies” lane 15A indicates the name of a transformant into which a multiple number of copies (more than two copies) of the miraculin gene were introduced. Lane 3-1 indicates a non-transformant that was not transformed with the miraculin gene. Samples of lanes other than lanes 11A, 20-1, and 14-3 were all from diploid tomato plants. The arrow indicates the presence of a dimer-sized miraculin protein.

FIG. 7 is a photograph showing the results of Western blot analysis, in which protein extracts from recombinant (transgenic) tomato leaves were developed by SDS-PAGE under reducing conditions, followed by detection of miraculin. Wt indicates a wild-type tomato plant (into which no miraculin gene was introduced). M indicates a miraculin protein isolated and purified from miraculin fruit. Lane names 2A, 3A, 6A, and 14C each indicate the names of recombinants prepared by the introduction of pBI35SMIR.

FIG. 8 shows positions of primers that were designed for amplification of the miraculin gene. Positions to which primers respectively hybridize are indicated with arrows on the cDNA sequence (Accession No. D38598) of the miraculin gene. Each bent tail portion of an arrow indicates an adaptor sequence contained in the 5′ terminus of a primer. Sequences enclosed with frames are sequences encoding signal-like peptide portions of the miraculin protein (i.e., signal-like peptide-coding sequences).

FIG. 9 shows photographs showing electrophoresis of PCR-amplified products of the miraculin gene. FIG. 9(A) shows the result with PCR products amplified using a primer set M1 (with amplification of a signal-like peptide-coding sequence). FIG. 9(B) shows the result with PCR products amplified using a primer set M2 (without amplification of a signal-like peptide-coding sequence).

FIG. 10 is a map of a pBigs2113SF vector into which a PCR-amplified fragment of the miraculin gene was inserted.

FIG. 11 is a photograph showing the results of Western blot analysis. The results show the expression of miraculin in T2 plants of the recombinant Arabidopsis thaliana plants of group M1, into which the amplified products with a signal-like peptide-coding sequence were introduced. Western blot analysis was performed for protein extracts that had been obtained from leaf samples and developed under non-reducing conditions. PM indicates a prestained protein marker. MIR indicates miraculin that was isolated and purified from miraculin fruit. Wt indicates an Arabidopsis thaliana plant into which no miraculin gene was introduced. Lane names M1-1-1 to M1-9-2 indicate the names of T2 plants of group M1. Arrows on the right side in the photograph indicate bands showing miraculin dimers and bands showing miraculin monomers.

FIG. 12 is a photograph showing the results of Western blot analysis. The results show the expression of miraculin in recombinant Arabidopsis thaliana plants of group M1, into which the amplified products with a signal-like peptide-coding sequence were introduced, or recombinant Arabidopsis thaliana plants of group M2, into which the amplified products without signal-like peptide-coding sequences were introduced. Western blot analysis was performed for protein extracts that had been obtained from leaf samples and then developed under non-reducing conditions. PM indicates a prestained protein marker. MIR indicates miraculin that was isolated and purified from miraculin fruit. Wt indicates a wild-type Arabidopsis thaliana plant into which no miraculin gene was introduced. Lane names M1-4-4 and M1-9-4 indicate the names of plant individuals into which the amplified products with a signal-like peptide-coding sequence were introduced. Lane names M2-1 and M2-6 indicate the names of plant individuals into which the amplified products without signal-like peptide-coding sequences were introduced.

### BEST MODE FOR CARRYING OUT THE INVENTION

1. Miraculin Gene and its Isolation

Miraculin contained in the fruit of a miracle fruit plant (Richadella dulcifica) is currently used industrially. However, the miracle fruit plant is- a plant of the tropics. Cultivation of this plant in non-tropical areas is extremely difficult, so that miraculin is not supplied in sufficient amounts. The present inventors have considered that an effective means for addressing the problem is to introduce the miraculin gene into a plant that can be cultivated all over the world using established cultivation technology for the plant and to cause the plant to produce the miraculin protein.

The present inventors have obtained the isolated miraculin cDNA from miracle fruit based on the nucleotide sequence information of the miraculin gene registered in the DNA database disclosed to the public. The cDNA of the miraculin gene used in the present invention can be prepared by preparing primers based on the cDNA sequence that is available under Accession No. D39598 from an international nucleotide sequence database or the nucleotide sequence information of SEQ ID NO: 1 listed in the sequence listing, and then amplifying by RT-PCR using the primers and the total RNA derived from miracle fruit as a template (see Example 1), for example.

However, the miraculin gene to be used in the present invention is not limited to cDNA obtained by such method and may be any isolated nucleic acid encoding the miraculin protein. “Miraculin” in the present invention refers to a protein having so-called taste-modifying activity that has been originally isolated from the fruit of the miracle fruit plant. “Taste-modifying activity” (also called “sense-of-taste-modifying activity”) possessed by miraculin refers to mainly activity that modifies sourness to sweetness; that is, activity that modifies the sense of taste, so that a person experiences a sweet taste when he or she places a sour material into his or her mouth after eating miraculin. The miraculin gene of the present invention is preferably a miraculin-encoding nucleic acid derived from the miracle fruit plant (Richadella dulcifica). An example of the miraculin gene is a gene comprising the nucleotide sequence as shown in SEQ ID NO: 1, or more preferablely a gene encoding the amino acid sequence as shown in SEQ ID NO: 2, wherein the amino acid sequence is encoded by the nucleotide sequence of SEQ ID NO: 1. Further examples of the miraculin gene of the present invention include miraculin gene homologs derived from plants other than the miracle fruit plant (e.g., LeMir (DDBJ/EMBL/GenBank Accession No. U70076) of a tomato plant (Lycopersicon esculentum) and soybean trypsin inhibitors A and C (Kunitz) of a soybean plant). The miraculin gene to be used in the present invention may be a gene comprising a nucleotide sequence derived from the nucleotide sequence of a natural miraculin gene (SEQ ID NO: 1, for example) by one or a plurality of mutations such as substitutions, deletions, additions and/ insertions, as long as the gene encodes a protein having the taste-modifying activity of miraculin (miraculin activity). The miraculin gene of the present invention may also be a gene encoding a protein that comprises an amino acid sequence derived from the amino acid sequence of natural miraculin having taste-modifying activity (SEQ ID NO: 2, for example) by deletion, substitution, or addition of one or several (2 to 9, preferably 2 to 5) amino acids and has taste-modifying activity. Furthermore, the miraculin gene of the present invention may also be a gene comprising DNA that hybridizes under stringent conditions to DNA having the nucleotide sequence of SEQ ID NO: 1 and encodes a protein having taste-modifying activity. Alternatively, the miraculin gene of the present invention may also be a gene comprising a nucleotide sequence that has at least 85% or more, and preferably 90% or more identity with the nucleotide sequence as shown in SEQ ID NO: 1 and encoding a protein that has taste-modifying activity. The miraculin gene of the present invention may also be a gene encoding a protein that comprises an amino acid sequence having at least 60% or more, preferably 70% or more, and further preferably 85% or more identity with the amino acid sequence as shown in SEQ ID NO: 2 and has taste-modifying activity.

In the present invention, the expression “stringent conditions” refers to conditions under which a nucleic acid hybrid is specifically formed. Specifically, such stringent conditions refer to reaction conditions in which a sodium salt concentration ranges from 15 mM to 750 mM, preferably 50 mM to 750 mM, and more preferably 300 mM to 750 mM, temperature ranges from 25° C. to 70° C., and more preferably 55° C. to 65° C., and formamide concentration ranges from 0% to 50%, and more preferably 35% to 45%, for example. Under more preferred stringent conditions, post-hybridization filter-washing conditions are conditions in which a sodium salt concentration ranges from 15 mM to 600 mM, preferably 50 mM to 600 mM, and more preferably 300 mM to 600 mM, and temperature ranges from 50° C. to 70° C., preferably 55° C. to 70° C., and more preferably 60° C. to 65° C.

The various above described miraculin genes can also be obtained by persons skilled in the art by mutagenesis of an existing miraculin gene using well-known technology such as a site-directed mutagenesis method (Ausubel et al., 1999, Current Protocols in Molecular Biology).

In the present invention, a “gene” may be DNA or RNA. Examples of such DNA include at least genomic DNA and cDNA. Examples of such RNA include mRNA and the like. The miraculin gene of the present invention may also contain an untranslated region (UTR) in addition to a CDS sequence (ranging from an initiation codon to a termination codon) of miraculin gene.

The above described nucleic acid fragments such as DNA or the like containing the miraculin gene of the present invention can be obtained from a nucleic acid extracted from an organism having the miraculin gene by persons skilled in the art according to a standard method on the basis of the known sequence of the miraculin gene.

In one embodiment of the present invention, a DNA fragment containing the miraculin gene of the present invention can be obtained as cDNA through nucleic acid amplification by RT-PCR using as a template mRNA extracted by a standard method from the tissue of a plant capable of expressing miraculin and primers designed to enable amplification of the full-length sequence of the miraculin gene (ranging from the initiation codon to the termination codon).

It is convenient to clone the thus obtained DNA fragment containing the miraculin gene into a vector. For example, the miraculin gene is inserted into a known expression vector such as an Escherichia coli expression vector (e.g., pUC19). The vector is then introduced into a host such as Escherichia coli (e.g., BL21 strain), so that the gene can be expressed within the host. The DNA fragment containing the miraculin gene can also be inserted downstream of an overexpression (or high expression) promoter for a plant within a recombinant expression vector. Another suitable recombinant expression vector is a vector wherein the DNA fragment containing the miraculin gene is inserted downstream of an enhancer and an overexpression (high expression) promoter for a plant. In this case, for example, both termini of the DNA fragment containing the miraculin gene are cleaved with appropriate restriction enzymes and then the cleaved fragment is inserted in-frame and ligated to an appropriate restriction enzyme site located downstream of an overexpression promoter within the vector. When an Agrobacterium method is employed for gene transfer to a plant, the miraculin gene is preferably incorporated into a vector appropriate for the Agrobacterium method, such as a plasmid vector derived from Agrobacterium (e.g., pBI101 or pBI121) or a modified expression vector therefrom. For example, a marker gene portion of a known vector (generally used pBI121, pBI221, or the like) that has been developed for expression in a plant is excised using restriction enzymes, and the miraculin gene instead of the marker gene is then incorporated into the vector. Hence, a vector for the Agrobacterium method can be constructed. With this method, the miraculin gene is generally placed under the control of an overexpression (high expression) promoter.

Moreover, it is preferable to verify the sequence of the thus obtained DNA fragment containing the miraculin gene through determination of the nucleotide sequence. A nucleotide sequence can be determined by a known technique such as Maxam-Gilbert chemical modification method or a dideoxynucleotide chain termination method. In general, a nucleotide sequence is determined using an automated DNA sequencing instrument (e.g., ABI PRISM377XL DNA sequencer).

Molecular biological and biochemical experimental protocols employed in the present invention, such as mRNA preparation, cDNA preparation (RT-PCR), PCR, ligation into vectors, transformation of cells, DNA sequencing, primer synthesis, and protein extraction can be performed according to descriptions in general laboratory manuals. An example of such a laboratory manual is Sambrook et al., Molecular Cloning, A Laboratory Manual, 2001, Eds., Sambrook, J. & Russell, D. W., Cold Spring Harbor Laboratory Press.
2. Preparation of Recombinant Plant into which a Miraculin Gene is Introduced

According to the present invention, a transgenic plant having taste-modifying activity can be produced by introducing the miraculin gene obtained as described above into a plant.

For example, as described later in Example 2, an expression vector for the Agrobacterium method in which the miraculin gene has been incorporated is introduced into agrobacteria. The vector can be used for gene transfer into plant cells with the use of the Agrobacterium method. In the present invention, a vector derived from pBI121 can be suitably used for the Agrobacterium method. The plant cells into which such a vector is introduced may be in any form of plant tissues, organs, calli, and the like.

In the present invention, plants into which the miraculin gene is introduced are not limited and may be unicellular algae, Gymnospermae, dicotyledoneae, or monocotyledons. In the present invention, more preferablely, a plant into which the miraculin gene is introduced is a crop plant such as lettuce, tomato, strawberry, or Arabidopsis thaliana. It has been reported that gene transfer based on the Agrobacterium method is broadly used for a plant such as rice, tomato, melon, or lettuce, although they differ in terms of gene transfer efficiency. In addition, in the present invention, it is particularly useful to introduce the miraculin gene into a diploid plant, as described later.

Methods for introducing the miraculin gene into plant cells are not limited to the Agrobacterium method. A direct method such as an electroporation method or a particle gun method can also be employed. For details about these plant transformation methods, see descriptions in general textbooks such as “New Edition, Experimental Protocols for Model Plants, Genetic Techniques to Genomic Analysis” (under the supervision of Isao Shimamoto and Kiyotaka Okada, (2001) Shujunsha Co., Ltd.) or literature such as Hiei Y. et al., “Efficient Transformation of Rice (Oryza sativa L.) Mediated by Agrobacterium and Sequence Analysis of the Boundaries of the T-DNA” Plant J. (1994) 6, 271-282.

Plant cells into which a vector has been introduced by the Agrobacterium method can be generally cultured in a tissue culture medium containing an antibiotic for selecting transformed cells, an antibiotic for eliminating agrobacteria, and a plant hormone for regeneration of whole plants (e.g., auxin and cytokinin), and thereby whole plants can be regenerated. Plant cells into which a vector has been introduced by an electroporation method or a particle gun method are generally cultured in a tissue sulture medium containing an antibiotic for selecting transformed cells and a plant hormone for regeneration of whole plants (e.g., auxin and cytokinin), and thereby whole plants can be regenerated.

For example, the following method can be employed for lettuce plants. The miraculin gene can be introduced into lettuce plants by a leaf disc method using Agrobacterium strain GV2260. The Agrobacterium cells harboring the miraculin gene are shake-cultured overnight in LB media with 100 mg/L kanamycin. After washing by centrifugation, the cells are suspended in MS liquid media with 200 μM acetosyringone and 10 μM mercaptoethanol at an OD₆₀₀ of 0.1. Sterile lettuce leaf sections at day 5 after seeding are immersed in the thus obtained Agrobacterium solutions. The lettuce leaf sections subjected to the infection with the Agrobacterium are co-cultured for 3 days in MS media with 1 mg/L benzyladenine (BA) and 0.1 mg/L naphthalenacetic acid (NAA). Subsequently, the sections are transferred to selection MS media with 0.1 mg/L BA, 0.1 mg/L NAA, and 100 mg/L kanamycin and then cultured while changing the media every 2 weeks. Shoots that have differentiated are transferred to MS media with 0.01 mg/L BA, 0.05 mg/L NAA, and 50 mg/L kanamycin. Shoots that have grown about 1 to 3 cm high are transferred to rooting MS media with 50 mg/L kanamycin. Confirmation of transgene is performed for the thus obtained regenerated plant individuals, so as to confirm transformants.

Furthermore, a method described in literature (Ohyama et al., 1995, Plant Cell Physiology, 36: 369-376) can be employed for tomato plants, for example. Specifically, tomato seeds are aseptically seeded on MS media and caused to germinate at 25° C. under 16-hour lighting (approximately 4,000 lux). Each cotyledon of seedlings that have germinated is divided into two portions, and then they are inoculated with Agrobacterium cells (Agrobacterium tumefaciens) (e.g., the LBA4404 strain) having a gene to be introduced are then introduced, followed by co-culturing them for 2 days on MS media with 1 mg/L zeatin. Subsequently, the cells are cultured for 1 week on MS media with 1 mg/L zeatin and 200 mg/L carbenicillin for bacterial elimination. Furthermore, the cells are subcultured every 2 weeks on MS media with 1 mg/L zeatin, 200 mg/L carbenicillin, and 100 mg/L kanamycin. Shoots that have grown are transferred and subcultured on MS media with 200 mg/L carbenicillin and 50 mg/L kanamycin, so as to cause rooting. Confirmation of transgene is performed for the thus obtained regenerated plant individuals, so as to confirm transformants.

A transgene can be confirmed by the following method. Incorporation of a transgene into a nucleus can be confirmed by Southern analysis performed on genomic DNA. Transcription of the incorporated gene within a nucleus to mRNA can be confirmed by Northern analysis. Translation of mRNA to miraculin can be confirmed by Western analysis (see Example 4). A miraculin-recognizing specific antibody to be used for Western analysis can be prepared by a known method using miraculin that is expressed by Escherichia coli as an antigen (see Example 5).

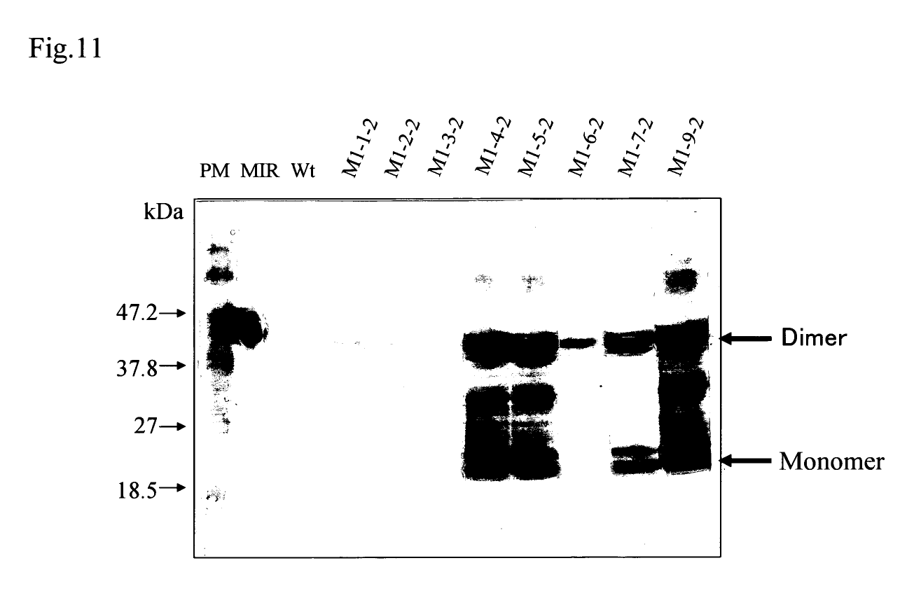

Miraculin proteins produced in recombinant plants into which the miraculin gene of the present invention has been introduced may form dimers. This can be confirmed by Western analysis using a miraculin-specific antibody, for a protein solution that has been developed by SDS-PAGE under non-reducing conditions (e.g., see FIG. 11). Miraculin protein dimers are active (Kurihara, 1992, Critical Reviews in Food Science and Nutrition, 32(3): 231-252), but miraculin protein monomers are inactive. Accordingly, it has been demonstrated that the miraculin gene introduced into the recombinant plant of the present invention enables accumulation of active miraculin.

The recombinant plant of the present invention into which the miraculin gene has been introduced produces miraculin, and thereby exhibiting taste-modifying activity. Specifically, when a subject (preferably, a human) places the recombinant plant of the present invention in his or her mouth, and preferably chews it well, and then places a sour material in his or her mouth, the subject experiences a sweet taste instead of a sour taste.

Accordingly, the miraculin activity (i.e., taste-modifying activity) produced by such recombinant plant can be assayed by a bioassay method as described in Examples 6 to 8. With a direct method, such a taste-modifying effect can be assayed by placing a recombinant plant producing miraculin directly in human mouth and then placing a sour material (e.g., citric acid or lemon juice) in the mouth. With an extraction method, such effect can be assayed by extracting miraculin by a known method from such recombinant plant, placing the extract in a subject's mouth, and then placing a sour material (e.g., citric acid or lemon juice) in the mouth. In these assays, when a subject experiences a sweet taste instead of a sour taste concerning the sour material placed in his or her mouth, it can be concluded that miraculin produced in the recombinant plant has such activity.

Regarding lettuce, tomato, and the like, cultivation techniques and distribution techniques have been developed to a great extent, and the plants can be stably produced throughout the year in almost all areas in the world. Hence, these plants are extremely appropriate hosts for making miraculin producing recombinant plants. To put it another way, plants into which the miraculin gene is introduced should not be plants that cannot be easily cultivated in areas other than specific areas, such as the miracle fruit plant (Richadella dulcifica), in terms of ease of cultivation. However, examples of a useful plant into which the miraculin gene may be introduced and in which it is expressed herein are not limited thereto, and include all plants that can serve as foodstuffs.

In addition, the present inventors have found as also described later in the Examples that a diploid transformed (transgenic) plant obtained by the introduction of the miraculin gene into a diploid plant possesses particularly high ability to produce miraculin. The present inventors have confirmed that a diploid plant into which the miraculin gene has been introduced accumulates miraculin in an active dimeric form and that miraculin has sweetness-inducing activity. Furthermore, the present inventors have also found that decreased expression levels are unlikely to occur in diploid plant cells into which the miraculin gene has been-introducecd. Accordingly, it has been demonstrated that a diploid plant is particularly appropriate for high expression of the miraculin gene. Diploid transgenic plants into which the miraculin gene has been introduced can stably produce active miraculin in large amounts, so that they are particularly useful for the large-scale production of miraculin.

In terms of ease of cultivation, it is further suitable to use a diploid crop plant (cultivated species) as a diploid plant into which the miraculin gene is introduced. Examples of the above diploid plants include, but are not limited to, diploid plants such as those of tomato, cabbage, Chinese cabbage, rice, corn, soybean and the like.

In the case of animals, almost no polyploids having a chromosome number that is more than diploid are known, excluding some cases of amphibians or fish. However, it is known among plants that more than one type of polyploids is often found within the same species. Polyploid plants can generate advantageous traits in terms of cultivation, such as the bearing of larger fruits, so that many of them are used advantageously as crops. Polyploid crop plants that are currently used are both natural and artificially produced plants. Examples of ploidies in crop plants include, in addition to a wheat diploid cultivar (having double sets of chromosomes), a tetraploid cultivar. (having 4 sets of chromosomes) and a hexaploid cultivar (having 6 sets of chromosomes). Meanwhile, the cultivated species of sweet potato is a hexaploid, cultivated tobacco is an allotetraploid (a polyploid resulting from a heterologous combination of each 2 genomic sets; also referred to as an amphidiploid). However, for the purpose of performing highly efficient recombinant production of miraculin in the present invention, a plant into which the miraculin gene is introduced is preferably not a polyploid (e.g., a tetraploid, a hexaploid, or an amphidiploid), but a diploid as described above. In the present invention, “diploid” means a plant having double genomic sets (a genomic set means one set of chromosomes).

To produce transgenic plants expressing miraculin at high levels, it is more preferable in the present invention to introduce the miraculin gene into diploid plants and to further select diploid plants thereamong to ensure obtainment of diploid transgenic plants. This is because even when the miraculin gene is introduced into diploid plant cells, chromosomes are often naturally polyploidized to tetraploids or the like in a cell culture step such as subculture (see Examples). To select diploid plants, it is preferable to selectively grow the cells into which the miraculin gene has been introduced under marker selection or the like, and optionally grow the cells to whole plants, subsequently determine ploidy for the grown plant cells (in the case of whole plants, cells of the leaves or roots are preferable) using a ploidy test method known by persons skilled in the art, and then select diploid cells. As a non-limited example, cells of interest derived from leaves of the whole plants can be tested for ploidy using a flow cytometer (e.g., Flow cytometer (Partec)). In this test, the ploidy of whole plants into which the miraculin gene has been introduced can be determined by identifying a observed peak therefor compared to one for a sample derived from a plant of which ploidy has been known, as a control. The present invention also includes a method for producing transgenic plants that express miraculin at high expression levels, comprising introducing the miraculin gene into diploid plants and then selecting diploid plants among the whole plants into which the gene has been introduced.

The terms “transgenic plant,” “recombinant plant,” and “(plant) transformant” used herein are interchangeably used to refer to plants into which the miraculin gene has been introduced as a foreign gene. The terms “transgenic plant,” “recombinant plant,” and “(plant) transformant” mean not only whole plants, but also plant cells expressing a transgene or harboring a transgene which can be expressed, as well as plant parts containing such plant cells, including organs (e.g., leaves, flowers, stems, roots, and seeds), tissues (e.g., epidermis, phloem, parenchyma, xylem, vascular bundles, palisade tissues, and spongy tissues), and cultured cells (e.g., calli) that contain such plant cells, for example. “Transgenic plants,” “recombinant plants,” and “(plant) transformants” of the present invention also include progeny plants being whole plants regenerated from the transfected plant cells or calli containing such plant cells, and parts thereof.

Once a recombinant plant producing miraculin has been produced, the propagation materials (e.g., fruits and seeds) thereof can be easily obtained. Moreover, it becomes possible to produce recombinant plants producing miraculin in large amounts with the use of the thus obtained propagation materials. Furthermore, persons skilled in the art can obtain miraculin protein in large amounts from such miraculin-producing recombinant plants using a known protein extraction and purification method. Therefore, the present invention also relates to a method for producing miraculin in large amounts, comprising extracting proteins containing miraculin from the recombinant plants and preferably further separating miraculin therefrom.
3. Food or Drink and Pharmaceutical Preparations

The present invention also relates to a food or drink and a food additive containing miraculin produced by the recombinant plant of the present invention. The present invention further relates to a pharmaceutical preparation containing miraculin as an active ingredient, which is produced by the recombinant plant of the present invention.

Miraculin that is produced by the recombinant plant of the present invention has taste-modifying activity by which sourness is converted to sweetness. Accordingly, a food or drink, a food additive, or a pharmaceutical preparation (e.g., a therapeutic drug, a prophylactic drug, or a life-improving drug) containing miraculin that is produced by the recombinant plant of the present invention is useful for preventing diabetes or promoting the therapeutic effects on diabetic patients (e.g., for alleviating stress during dietary therapy), for non-limited examples.

The whole plant of the recombinant plant of the present invention, which expresses the introduced miraculin gene, can be directly used as a food. Alternatively, a part of such whole plant can also be used as a food or a food additive. Miraculin protein or a protein fraction containing such miraculin that has been extracted from the whole plant expressing the introduced miraculin gene or parts of the same (e.g., leaves) can also be used in a food or drink, a food additive, or a pharmaceutical preparation. One embodiment of the present invention concerning a food or drink, a food additive, or a pharmaceutical preparation that contains miraculin that is produced by the recombinant plant of the present invention is a food or drink, a food additive, or a pharmaceutical preparation that contains the whole plant of the miraculin-expressing recombinant plant of the present invention, a part of such whole plant (e.g., leaves or seeds), or a protein extract containing miraculin from such whole plant or parts of the same.

In this description, examples of a “food or drink” include, but are not limited to, a beverage, a food, and a functional food. In the food or drink of the present invention, it is preferable to add a sour material (e.g., lemon juice or citric acid) in addition to miraculin produced by the recombinant plant of the present invention. Since miraculin has the property of converting sourness to sweetness, the food or drink of the present invention preferably has reduced sugar content, instead of being supplemented with a sour material. Examples of such food or drink to which miraculin produced by the recombinant plant of the present invention is added include, but are not particularly limited to, beverages such as fruit or vegetable beverages (e.g., a beverage containing fruit juice such as orange, apple, or grape juice, or vegetable juice such as tomato or carrot juice), alcoholic beverages (e.g., beers, sparkling liquor, and wine), carbonated beverages, soft drinks, fermented milk (e.g., yogurt), lactic acid bacteria beverages, milk beverages (e.g., coffee milk and fruit milk), and tea-based beverages (e.g., green tea, black tea, and oolong tea). Regarding methods for producing various types of beverages and the like, see, existing books of reference such as “Latest Soft Drinks” (2003) (KORIN PUBLISHING CO., LTD.).

A food to which miraculin that is produced by the recombinant plant of the present invention is added is not particularly limited and may also be a fresh food or a processed food. Examples of such food include various foods such as pudding, jellies, ice creams, cakes, candies, pasta, udon (Japanese wheat noodle), fish sausages, hams, soy sauce, dressings, mayonnaise, tofu, soups, bread, fillets, processed meat, vegetables, and mushrooms.

A functional food is particularly preferable as a food or drink containing miraculin that is produced by the recombinant plant of the present invention. “Functional food” in the present invention means a food that exerts particular functionality upon living bodies. Examples of such functional food include foods for specified health use (including qualified FOSHU. (food for specified health use; also called Tokuho in Japan)), foods with health claims including foods with nutrient function claims, foods for special uses, nutritional supplements, health supplements, supplements (e.g., supplements in various dosage forms, such as tablets, coated tablets, sugar-coated tablets, capsules, and liquid drugs), and all so-called health foods such as beauty foods (e.g., diet foods). Examples of the functional food of the present invention also include health foods to which “health claims” are applied under Codex Alimentarius (JOINT FAO/WHO CODEX ALIMENTARIUS COMMISSION).

More specific examples of preferable functional food of the present invention include foods for special use such as patient foods, powdered milk for pregnant and parturient/lactating women, and foods for elderly.

Preferable examples of functional foods containing miraculin that is produced by the recombinant plant of the present invention include foods with health claims. The system concerning foods with health claims in Japan is intended not only for general foods, but also for foods formulated into forms such as tablets and capsules. Under the system, foods with health claims are two types: foods for specified health use (approved FOSHU) and foods with nutrient function claims (standardized FOSHU). There are new types such as qualified FOSHU (food for specified health use; also called Tokuho).

The functional food of the present invention has activity to convert sourness to sweetness. Therefore, the functional food of the present invention has an effect of promoting the therapeutic effects for obesity or diabetes in terms of daily life to enable persons afflicted with obesity or diabetic patients to be able to experience sweet tastes without ingesting sugar during dietary therapy. The functional food of the present invention (preferably, a food for specified health use or qualified FOSHU) may be provided with a description or labeling that shows the fact that such food enables comfortable suppression of the amount of sugar intake. Such description or labeling may be approved under the system for foods with health claims. Examples of descriptions or labelings for the functional foods of the present invention are “useful for reduction of sugar intake” and “useful for improving dietary habits of a person who wants to lose weight.”

The functional food of the present invention may be in form of drug formulation such as a solid formulation (e.g., tablets, granules, powders, pills, and capsules), a liquid formulation (e.g., liquids, suspensions, and syrups), or a gel formulation. Furthermore, the functional food of the present invention may also be in the form of a general food or drink (e.g., beverages, powdered tea, and sweets).

The content of miraculin that is produced by the recombinant plant of the present invention in a food or drink is not particularly limited and may be varied depending on the case at hand. Specific miraculin content can be appropriately determined by persons skilled in the art in view of the type of a given food or drink and is expected taste or texture. However, in general the appropriate content of miraculin that is produced by the recombinant plant of the present invention ranges from 0.001% to 100% by weight, and in particular ranges from 0.1% to 100% by weight.

For production of the food or drink of the present invention, various types of additives that are conventionally used for foods or drinks can also be used. Examples of additives include, but are not limited to, color fixing agents, food coloring agents, flavoring agents, preservatives, emulsifiers, antioxidants, pH adjusters, chemical seasonings, thickeners, antifoaming agents, and binding agents. Furthermore, a functional material such as a Panax ginseng extract, a Siberian Ginseng extract, a Eucalyptus extract, or a du zhong tea extract can also be added.

The food or drink of the present invention is preferably intended for ingestion by mammals including humans, domestic animals, pet animals, experimental (test) animals, and the like, which are required to comfortably reduce their sugar intake. In particular, persons who want to lose weight, obese persons, diabetic patients, and the like who need to comfortably reduce their sugar intake, are preferable subjects who ingest the food or drink of the present invention. Persons who are likely to gain weight or experience diabetes and the like are also preferable subjects who may ingest the food or drink of the present invention.

The present invention also relates to a food additive containing miraculin that is produced by the recombinant plant of the present invention. In addition to miraculin, various conventionally used types of additives may further be added to the food additive of the present invention. The food additive of the present invention can be used for producing a food or drink containing miraculin that is produced by the above described recombinant plant of the present invention.

The present invention also relates to a pharmaceutical preparation containing as an active ingredient miraculin that is produced by the recombinant plant of the present invention.

A pharmaceutically acceptable carrier or an additive can also be incorporated into the pharmaceutical preparation of the present invention. Examples of. such carrier or additive include water, pharmaceutically acceptable organic solvents, collagen, polyvinyl alcohol, polyvinylpyrrolidone, pectin, xanthan gum, gum Arabic, casein, glycerin, propylene glycol, polyethylene glycol, paraffin, stearyl alcohol, human serum albumin, mannitol, lactose, and surfactants that are acceptable as pharmaceutical additives. Further examples of the same include artificial cellular constructs such as liposomes. Additives to be used herein are selected appropriately or in combination depending on the dosage form of a formulation. The pharmaceutical preparation of the present invention may further contain other pharmacological ingredients.

The pharmaceutical preparation of the present invention is basically presented in an oral formulation. The pharmaceutical preparation of the present invention is required to act within the oral cavity, so that the pharmaceutical preparation may be in the dosage form of a solid formulation (e.g., tablets, granules, powders, pills, buccals, and troches), a liquid formulation (e.g., gels, liquids, suspensions, and syrups), or the like.

The above oral solid formulation may also contain an additive that is generally used pharmaceutically, such as binders, excipients, lubricants, disintegrants, and wetting agents. Furthermore, the above oral liquid formulation may also contain an additive that is generally used pharamaceutically, such as stabilizers, buffering agents, correctives, preservatives, flavoring agents, and coloring agents.

The dose of the pharmaceutical preparation of the present invention differs depending on the age and body weight of a subject to which the drug is administered, administration routes, and the number of administration. The dose can be broadly varied as is necessary by persons skilled in the art.

Preferable subjects to which the pharmaceutical preparation of the present invention is administered are mammals including humans, domestic animals, pet animals, experimental (test) animals, and the like, which need to comfortably reduce their sugar intake. Particularly preferable subjects are persons who want to lose weight, obese persons, or diabetic patients who need to comfortably reduce their sugar intake. Persons who are likely to gain weight or experience diabetes and the like are also preferable subjects.

One specific suitable example of a “pharmaceutical preparation” of the present invention is an antidiabetic drug. Examples of such antidiabetic drug include agents for preventing/ameliorating diabetes. Examples of such agent for preventing/ameliorating diabetes include agents for improving the life of diabetic patients by which stress during dietary therapy are alleviated. Another specific suitable example of a “pharmaceutical preparation” of the present invention is an obesity drug. Examples of such obesity drug include agents for preventing/ameliorating obesity. Examples of such agents for preventing/ameliorating obesity include agents for improving the life of obese patients by which stresses during dietary therapy are alleviated. The term “agents for preventing/ameliorating” used herein means pharmaceutical preparations that exert effects of preventing the onset of a given disease and/or effects of ameliorating the symptoms and the like of a given disease.

### EXAMPLES

The present invention will be described more specifically based on the following examples. However, the present invention is not limited to these examples.

### Example 1

### Cloning and Sequencing Analysis of Miraculin Gene

In this experiment, a miracle fruit and the leaves thereof were used as source materials for cloning a miraculin gene. The gene was obtained by the following method. Total RNA was extracted from the miracle fruit and the leaves thereof using a phenol-SDS method and then cDNA was synthesized from them with a RT-PCR high kit (Toyobo). Miraculin cDNA was amplified by PCR using the thus obtained cDNA as a template and two specific primers that had been designed based on the cDNA sequence information of the miraculin gene (Matuda et al., 1995): a sense primer 5′-TTTTCTAGAATGAAGGAATTAACAATGCT-3′ (SEQ ID NO: 3) in which a restriction enzyme Xba I site had been added and an antisense primer 5′-TTTGAGCTCTTAGAAGTATACGGTTTTGT-3′ (SEQ ID NO: 4) in which restriction enzyme Sac I site had been added. The PCR was performed with heat denaturation at 95° C. for 5 minutes, followed by 40 cycles of 95° C. for 1 minute, 57° C. for 1 minute, and 72° C. for 2 minutes. The PCR products were cleaved with Xba I and Sac I and then subcloned into pUC19 (pUCMRL19). The nucleotide sequence of a cloned fragment was determined using a DNA Sequencer ABI310 and DNA Sequencing Kit Big Dye Terminator Cycle Sequencing Ready Reaction (Applied Biosystems, Tokyo). The sequence was verified to be identical to the miraculin sequence registered in the database (DDBJ accession No. D39598). Of the nucleotide sequence of the cloned fragment, the sequence from the initiation codon to the termination codon of the miraculin gene is shown in shown in SEQ ID NO: 1. The amino acid sequence of the miraculin protein encoded by the nucleotide sequence of SEQ ID NO: 1 is shown in SEQ ID NO: 2.

### Example 2

### Construction of Plant Expression Vectors and Introduction of the Same into Agrobacterium

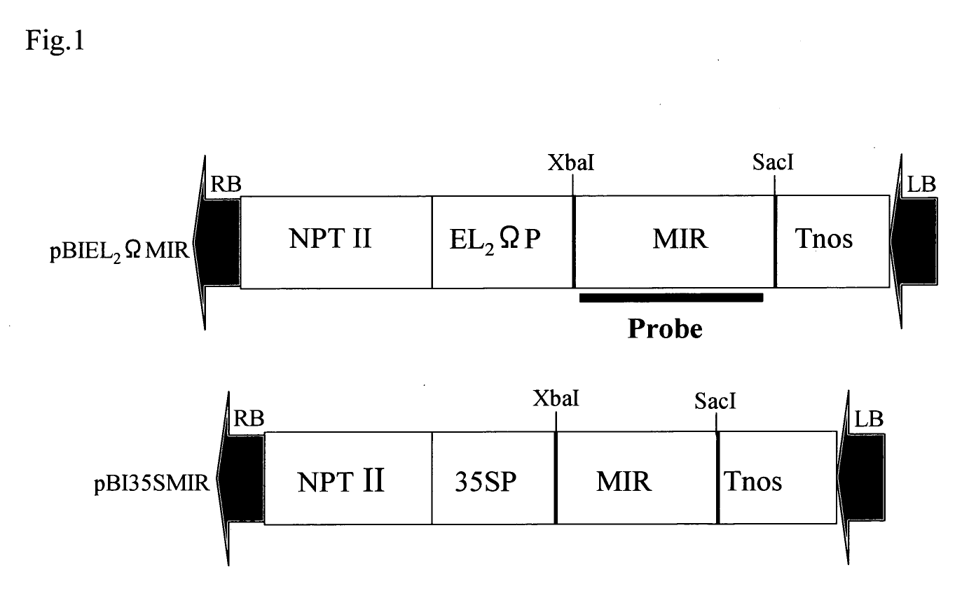

Plasmids were extracted from Escherichia coli (JM109) harboring the plasmid pUCMRL19, according to a standard method. The miraculin gene portion was excised using restriction enzymes Xba I and Sac I from the resulting plasmids. The portion was then cloned into GUS gene sites of a binary vector pBI121 for plant expression and a modified vector (high-expression vector) which had been made by modifying CaMV35S promoter region in pBI121. These resulting plasmids were designated pBI35SMIR and pBIEL₂ΩMIR, respectively (FIG. 1). Escherichia coli JM109 harboring each of the plasmids were cultured and maintained in LB media with 100 mg/L antibiotic kanamycin. Subsequently, Agrobacterium strain GV2260 was transformed with each of these 2 plasmids by electroporation. The thus transformed Agrobacterium strain GV2260 (into,which pBI35SMIR or pBIEL₂ΩMIR had been introduced) were cultured and maintained in LB media with 100 mg/L kanamycin.

### Example 3

### Preparation of Lettuce Recombinants

The miraculin gene was introduced into lettuce plants (cultivar ‘Kayser’; diploid) by a leaf disc method using the Agrobacterium strain GV2260 harboring pBI35SMIR or pBIEL₂ΩMIR. The transformed Agrobacterium cells were shake-cultured overnight in LB media with 100 mg/L kanamycin. After washing by centrifugation, the cells were suspended in an MS liquid medium with 200 μM acetosyringone and 10 μM mercaptoethanol at an OD₆₀₀ of 0.1. Sterile lettuce leaf sections at day 5 after seeding were immersed in the thus obtained Agrobacterium solutions. The lettuce leaf sections subjected to the infection with the Agrobacterium were co-cultured for 3 days in MS media with 1 mg/L benzyladenine (BA) and 0.1 mg/L naphthalenacetic acid (NAA). Subsequently, the sections were transferred to selection MS media with 0.1 mg/L BA, 0.1 mg/L NAA, and 100 mg/L kanamycin and then cultured while changing the media every 2 weeks. Shoots that had differentiated therein were transferred to MS media with 0.01 mg/L BA, 0.05 mg/L NAA, and 50 mg/L kanamycin. Then, shoots that had grown about 1 to 3 cm high were transferred to rooting MS media with 50 mg/L kanamycin. Transformed individuals were selected from individual plants that had formed roots in the media.

### Example 4

### Analysis of Recombinant Lettuce Plants

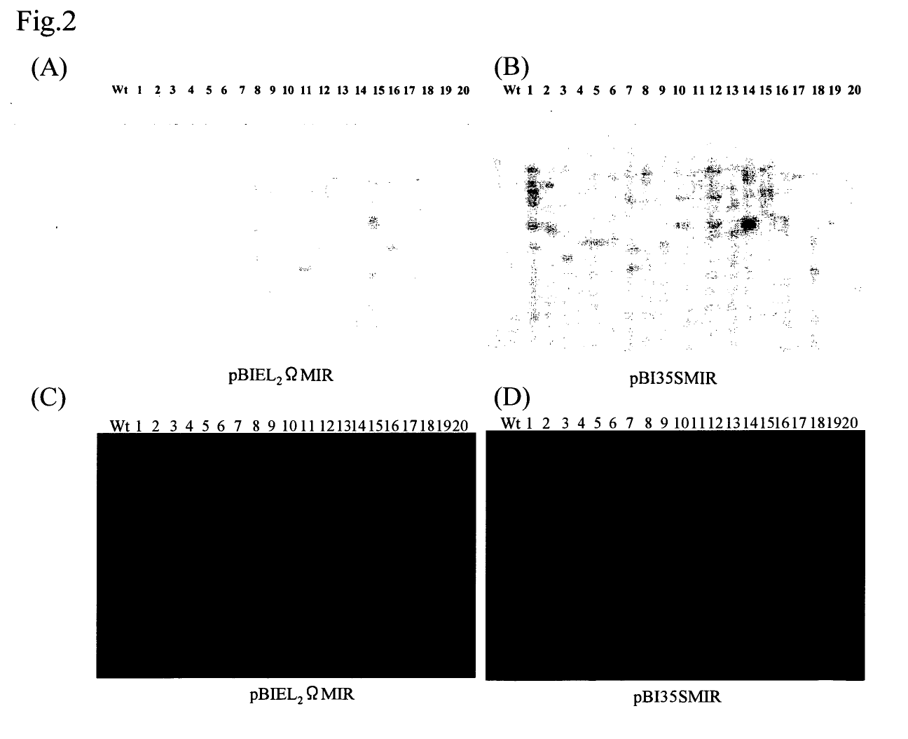

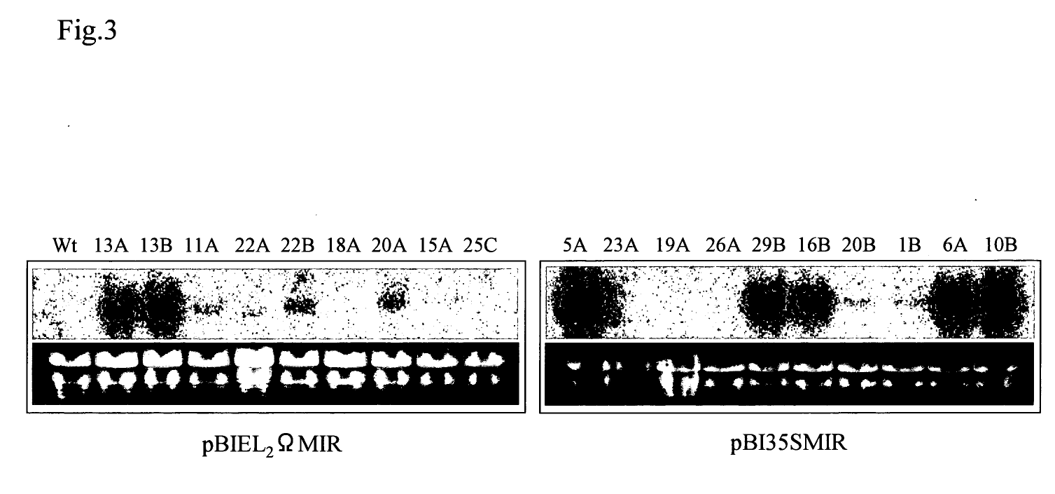

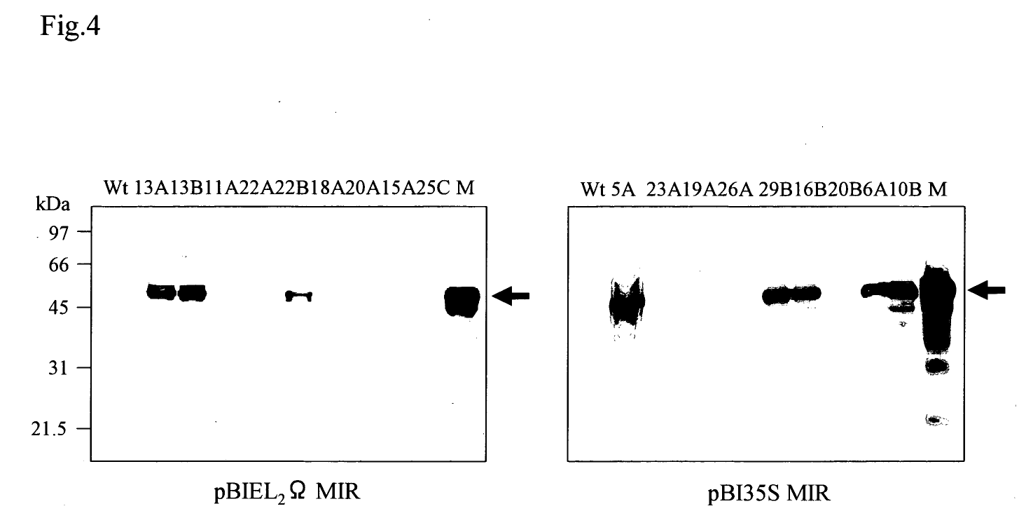

Genomic DNAs were extracted from the leaves from the resulting 40 individuals of transformant that had formed roots in the rooting media. 10 μg of the thus obtained genomic DNA was cleaved with Xba I, and subjected to Southern blot analysis with ³²P-labeled PCR-amplified fragments of the miraculin-coding region as a probe. As a result, signals were observed for all the plant individuals, so that the introduction of the miraculin gene thereinto was confirmed (FIG. 2). Subsequently, 19 plant individuals were selected randomly from the plant individuals for which the introduction of the miraculin gene thereinto had been confirmed, and then subjected to Northern analysis (FIG. 3). As a result, the expression of the miraculin gene was confirmed in 13 out of the examined 19 individuals. Next, the plant individuals, for which mRNA expression from the miraculin gene had been confirmed in Northern analysis, were subjected to Western analysis using a miraculin-recognizing specific antibody as prepared in Example 5 (FIG. 4). As a result, it was confirmed that in the recombinants that had shown a strong expression of the miraculin gene in Northern analysis, miraculin was also strongly expressed at the protein level.

### Example 5

### Preparation of Miraculin-Recognizing Specific Antibody

For the purpose of preparing a protein to be used as an antigen, PCR was performed using a sense primer 5′-TAGGATCCGATTCGGCACCCAATCCGGTT-3′ (SEQ ID NO: 5) in which a restriction enzyme BamH I site had been added, an antisense primer 5′-TTTGAGCTCTTAGAAGTATACGGTTTTGT-3′ (SEQ ID NO: 4) in which a restriction enzyme Sac I site had been added, and the full-length miraculin cDNA that had been subcloned into pUC19 (pUCMRL19) as a template. The PCR product (containing nucleotides 94 to 669 of the cDNA sequence of the miraculin gene available based on accession No. D38598) was treated with restriction enzymes BamH I and Sac I, and then cloned into Escherichia coli expression vector pQE30 (Qiagen, Chatsworth, Calif.) that enables the addition of a sequence encoding a 6×His tag to the N-terminus treated with the same restriction enzymes (pQEMRL30). The fusion protein obtained by inducing the expression thereof with IPTG (isopropyl-β-D-thiogalactopyranoside), was purified using a His-Trap column (Amersham Pharmacia Biotech, UK). Rabbits were immunized with the thus purified fusion protein as an antigen, thereby obtaining anti-serum (Scrum, Tokyo, Japan). For purification of an antigen-specific antibody from the anti-serum, a sense primer 5′-TAGGATCCGATTCGGCACCCAATCCGGTT-3′ (SEQ ID NO: 5) in which a restriction enzyme BamH I site had been added and an antisense primer 5′-TTCTCGAGGAACGCCGAGAAATTGATGTT-3′ (SEQ ID NO: 6) in which a restriction enzyme Xho I site had been added were designed, and PCR was performed using the thus designed primers and the full-length miraculin cDNA that had been subcloned into pUC19 (pUCMRL19) as a template. The PCR product (containing nucleotides 94 to 360 of the miraculin gene) was treated with restriction enzymes BamH I and Xho I, and then cloned into Escherichia coli expression vector pGEX4T-1 (Amersham Pharmacia Biotech, UK) for addition of a GST (glutathione S-transferase)-coding sequence to the N-terinus treated with the same restriction enzymes (pGEXMRL). The fusion protein obtained by inducing the expression thereof with IPTG (isopropyl-β-D-thiogalactopyranoside), was purified using a GST-trap column (Amersham Pharmacia Biotech, UK). An antigen-specific antibody was purified from the antiserum using an affinity column prepared by coupling the thus purified fusion protein to a CNBr-activated Sepharose 4 Fast Flow Column (Amersham Pharmacia Biotech, UK).

### Example 6

### Bioassay for Miraculin Produced by Recombinant Lettuce Plants

Bioassay was performed as follows. 10 g of the recombinant lettuce leaves, for which the expression of miraculin had been confirmed as described in Example 4, were washed well with water. Each subject chewed the leaves well in his or her mouth, applied them all over his or her tongue for 5 minutes and then expelled the leaves from his or her mouth. The subject rinsed his or her mouth with deionized water, and then tasted 0.02 M citric acid to compare the sweetness thereof that the subject had experienced upon tasting with those of 0.1 M to 1 M sucrose standard solutions. Furthermore, the protein was extracted by adding 10 mL of 0.5 M NaCl to 10 g of the recombinant lettuce leaves, for which the expression of miraculin had been confirmed, and then dialyzed against a 0.01 M NaHCO₃ solution. The thus obtained solution was used for a test. Its sweetness-inducing activity was assayed as follows. Each subject placed 5 mL of the protein solution in his or her mouth for 5 minutes to apply the solution well all over his or her tongue. The subject then expelled the solution, rinsed his or her mouth with deionized water, and then tasted 0.02 M citric acid. The sweetness intensity that the subject had experienced upon tasting was compared with those of 0.1 M to 1 M sucrose standard solutions.

In addition, in a manner similar to that described above, after 2 g of the recombinant lettuce leaves for which the expression of miraculin had been confirmed as described in Example 4 or non-recombinant lettuce leaves into which no miraculin gene had been introduced was washed well with water, each subject chewed the leaves well in his or her mouth, applied them all over his or her tongue for 5 minutes and then expelled the leaves from his or her mouth. For a bioassay, the subject rinsed his or her mouth with deionized water and then tasted 0.02 M citric acid to compare the sweetness thereof that the subject had experienced upon tasting with those of 0.1 M to 1 M sucrose standard solutions. For comparison, each subject applied one miracle fruit of which skin had been peeled all over his or her tongue for 5 minutes and then expelled the fruit from his or her mouth, rinsed his or her mouth with deionized water and then tasted 0.02 M citric acid to compare the sweetness thereof that the subject had experienced upon tasting with those of 0.1 M to 1 M sucrose standard solutions. Table I shows these results.

TABLE 1
Assay of the sweetness-inducing activity of the recombinant lettuce

| | | Sucrose concentration (M) | | | | | | | | | | |
|---|---|---|---|---|---|---|---|---|---|---|---|---|
| Subject | Sample | 0 | 0.1 | 0.2 | 0.3 | 0.4 | 0.5 | 0.6 | 0.7 | 0.8 | 0.9 | 1.0 |
| A | Miracle fruit | | | | | O | | | | | | |
| | Non-recombinant | O | | | | | | | | | | |
| | lettuce | | | | | | | | | | | |
| | Recombinant | | | | | O | | | | | | |
| | lettuce | | | | | | | | | | | |
| B | Miracle fruit | | | | | O | | | | | | |
| | Non-recombinant | O | | | | | | | | | | |
| | lettuce | | | | | | | | | | | |
| | Recombinant | | | | | O | | | | | | |
| | lettuce | | | | | | | | | | | |
| C | Miracle fruit | | | | | O | | | | | | |
| | Non-recombinant | O | | | | | | | | | | |
| | lettuce | | | | | | | | | | | |
| | Recombinant | | | | | O | | | | | | |
| | lettuce | | | | | | | | | | | |
| D | Miracle fruit | | | | | O | | | | | | |
| | Non-recombinant | O | | | | | | | | | | |
| | lettuce | | | | | | | | | | | |
| | Recombinant | | | | | O | | | | | | |
| | lettuce | | | | | | | | | | | |

In Table 1, the sweetness of each sample is indicated with the capital letter “O” corresponding to a sucrose concentration at which a subject experienced a degree of sweetness equivalent to that of the sample. As described above, miraculin expressed in lettuce plants exerted sweetness-inducing activity to a degree equivalent to that of miraculin contained in the miracle fruit. In addition, the recombinant lettuce plants tended to show decreased expression levels of miraculin as their generation passed.

### Example 7

### Preparation of Recombinant Tomato Plants

The miraculin gene was introduced into tomato plants (cultivar ‘Moneymaker’; diploid) by a leaf disc method using Agrobacterium strain GV2260 harboring pBI35SMIR or pBIEL₂ΩMIR that had been prepared in Examples 1 and 2. The transformed Agrobacterium cells were shake-cultured overnight in LB media with 100 mg/L kanamycin. After washing by centrifugation, the cells were suspended in an MS liquid medium with 200 μM acetosyringone and 10 μM mercaptoethanol at an OD₆₀₀ of 0.1. Sterile tomato cotyledon sections at day 7 after seeding were immersed in the thus obtained Agrobacterium solutions. The tomato leaf sections subjected to the infection with the Agrobacterium were co-cultured for 3 days in MS media with 1.5 mg/L zeatin. Subsequently, the sections were transferred to selection MS media with 1 mg/L zeatin and 100 mg/L kanamycin and then cultured while changing the media every 2 weeks. Shoots that had grown were transferred to rooting MS media with 50 mg/L kanamycin. Transformed individuals were selected from individual plants that had formed roots in the media.

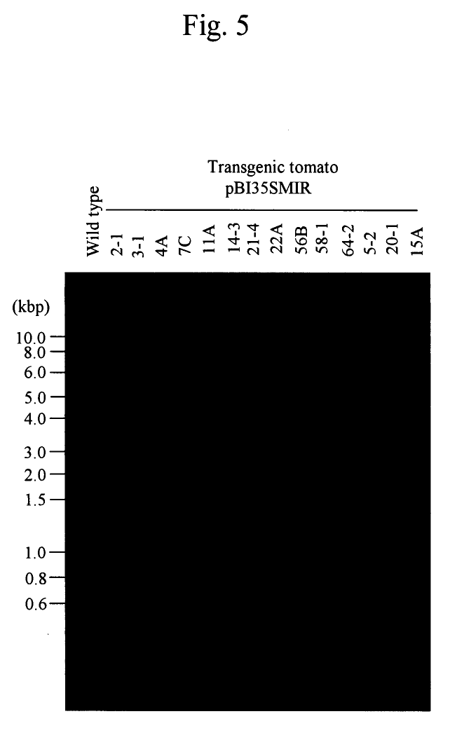

Genomic DNAs were extracted from the leaves of the transformed individuals that had formed roots in the rooting MS media and then subjected to Southern analysis, so as to test the transgene (FIG. 5). As a result, signals were observed for all of the plant individuals examined, so that the introduction of the miraculin gene thereinto was confirmed. The transgenic tomato plants into which the miraculin gene had been introduced were tested for ploidy using a flow cytometer (Partec).

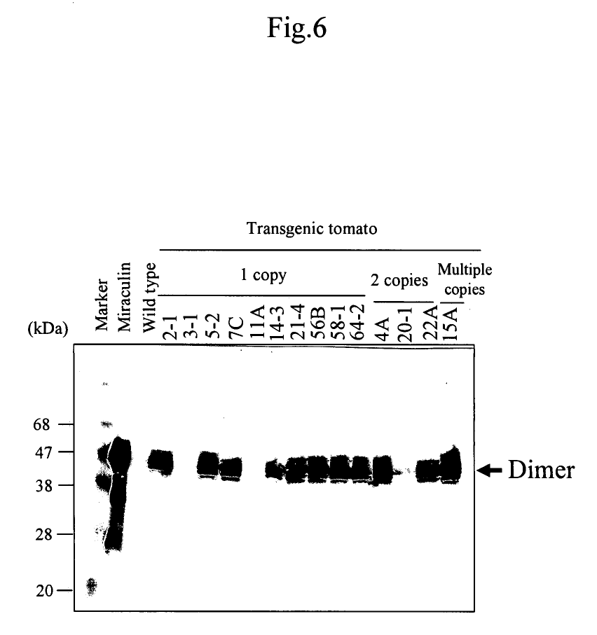

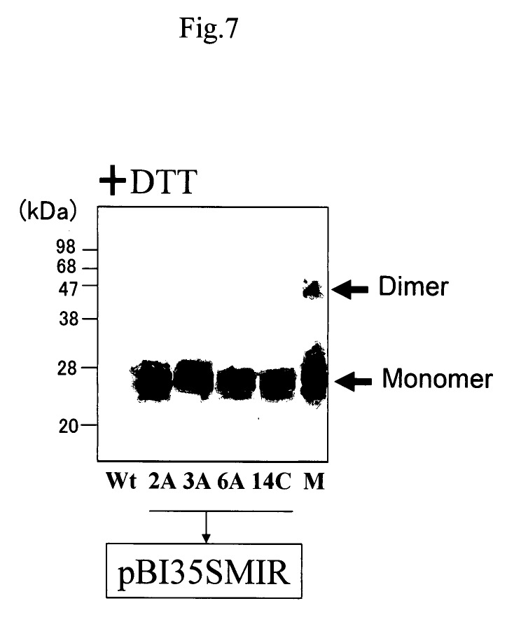

Subsequently, proteins were extracted from the leaves of the plant individuals for which the introduction of the miraculin gene thereto had been confirmed, and then subjected to Western analysis using the miraculin-specific antibody prepared in Example 5 by a standard method. SDS-PAGE was performed under non-reducing conditions, followed by Western blot analysis. FIG. 6 shows the results. In FIG. 6, lanes 11A, 20-1, and 14-3 represented samples derived from plant individuals that were tetraploids as determined by the flow cytometer. The samples represented in lanes other than these lanes were all derived from diploid individuals. Further, for all samples derived from diploid transgenic tomato plants, the dimer-size miraculin protein that was considered to be an active form was detected. In contrast, for samples each derived from tetraploid transgenic tomato lines 11A and 20-1, no miraculin was detected at all. For a sample derived from tetraploid transgenic tomato line 14-3, miraculin expression was confirmed, but the expression level was significantly lower than the expression levels of the diploid lines (a half thereof or less). Furthermore, samples derived from diploid transgenic tomato plants were subjected to SDS-PAGE under reducing conditions and then subjected to Western blot analysis. FIG. 7 shows the results. As shown in FIG. 7, miraculin was detected at the monomer-size. It is considered that under reducing conditions, dimers are separated into monomers. This means that, as shown in FIG. 7, miraculins that had formed dimers were prepared in the transgenic tomato plants. As in these results, strong miraculin expression was confirmed in the recombinant (transgenic) tomato plants.

Bioassay was performed as follows. 2 g of transgenic tomato pulp for which the expression of miraculin had been confirmed above or of non-recombinant tomato pulp into which no miraculin gene had been introduced were washed well with water. Each subject chewed the tomato pulp well in his or her mouth, applied it all over his or her tongue for 5 minutes, and then expelled the tomato pulp from his or her mouth. The subject rinsed his or her mouth with deionized water and then tasted 0.02 M citric acid to compare the sweetness thereof that the subject had experienced upon tasting with those of 0.1 M to 1 M sucrose standard solutions. For further comparison, each subject applied one miracle fruit of which skin had been peeled all over his or her tongue for 5 minutes and then expelled the fruit from his or her mouth, rinsed his or her mouth with deionized water, and then tasted 0.02 M citric acid to compare the sweetness thereof that the subject had experienced upon tasting with those of 0.1 M to 1 M sucrose standard solutions. Table 2 shows examples of the results.

TABLE 2
Evaluation of sweetness-inducing activity of recombinant tomato

| | | Sucrose concentration (M) | | | | | | | | | | |
|---|---|---|---|---|---|---|---|---|---|---|---|---|
| Subject | Sample | 0 | 0.1 | 0.2 | 0.3 | 0.4 | 0.5 | 0.6 | 0.7 | 0.8 | 0.9 | 1.0 |
| A | Miracle fruit | | | | | O | | | | | | |
| | Non-recombinant | O | | | | | | | | | | |
| | tomato | | | | | | | | | | | |
| | Recombinant | | | | | O | | | | | | |
| | tomato (diploid) | | | | | | | | | | | |
| B | Miracle fruit | | | | | O | | | | | | |
| | Non-recombinant | O | | | | | | | | | | |
| | tomato | | | | | | | | | | | |
| | Recombinant | | | | | O | | | | | | |
| | tomato (diploid) | | | | | | | | | | | |
| C | Miracle fruit | | | | | O | | | | | | |
| | Non-recombinant | O | | | | | | | | | | |
| | tomato | | | | | | | | | | | |
| | Recombinant | | | | | O | | | | | | |
| | tomato (diploid) | | | | | | | | | | | |
| D | Miracle fruit | | | | | O | | | | | | |
| | Non-recombinant | O | | | | | | | | | | |
| | tomato | | | | | | | | | | | |
| | Recombinant | | | | | O | | | | | | |
| | tomato (diploid) | | | | | | | | | | | |

In Table 2, the sweetness of each sample is indicated with the capital letter “O” corresponding to a sucrose concentration at which a subject experienced a degree of sweetness equivalent to that of the sample. As described above, miraculin expressed in tomato plants (diploids, in this case) exerted sweetness-inducing activity to a degree equivalent to that of miraculin contained in the miracle fruit.

In contrast, in the case of the pulp of a transgenic (recombinant) tomato line 14-3, which was a tetraploid line as a result of doubling, the sweetness that the subject had experienced upon tasting the citric acid was very weak. It was concluded that the sweetness-inducing activity of the transgenic tomato line 14-3 was significantly low.

### Example 8

### Preparation of Recombinant Arabidopsis thaliana Plants

mRNA was extracted from miracle fruit using the Dynabeads mRNA DIRECT Kit (DYNAL; Prod No. 610.11 & 610.12). cDNA was prepared by reverse transcription reaction using the Super Script First-Strand Synthesis System for RT-PCR (Invitrogen; Cat.No. 1 1904-018) and the mRNA as a template.

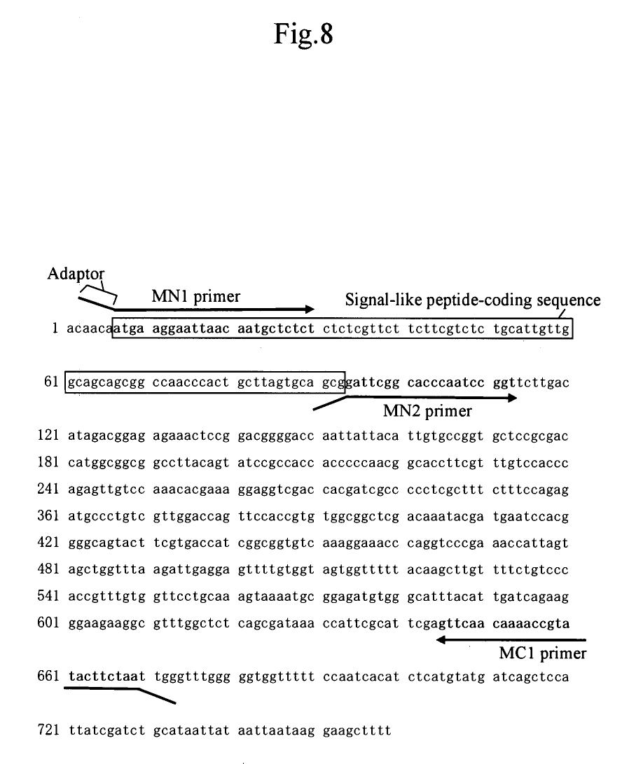

A DNA fragment containing the miraculin gene was amplified by PCR using the thus obtained cDNA as a template. Specifically, two primer sets were designed for PCR: a primer set M1 for amplification of a region that encodes a miraculin protein containing a signal-like sequence; and a primer set M2 for amplification of a region that encodes a miraculin protein portion and but contains no signal-like sequence and but to which ATG encoding methionine had been added (FIG. 8). Adaptors for cloning were added in these primers (FIG. 8).
Primer set M1 (the underlined portion indicates the 5′ terminal sequence of a signal-like peptide-coding sequence; amplification size of 753 bp):

Forward primer MN1:

(SEQ ID NO: 7)

5′-ATTACATTTTACATTCTACAACTACATCTAGAGGCC
AAATCGGCCatgaaggaattaac aatgctctct-3′
(capital letters: adaptor sequence)
Reverse primer MC1:

(SEQ ID NO: 8)

5′-CGAGCTCGCGGCCGCCCCGGGGATCCTCTAGAGGC
CCTTATGGCCttagaagtatacggttttgttgaac-3′
(capital letters: adaptor sequence)
Primer set M2 (containing no signal-like peptide-
coding sequence; amplification size of 669 bp)
Forward primer MN2:

(SEQ ID NO:9)

5′-ATTACATTTTACATTCTACAACTACATCTAGAGGCCA
AATCGGCCATGgattcgg cacccaatccggt-3′
(capital letters: adaptor sequence)
Reverse primer MC1:

(SEQ ID NO: 8)

5′-CGAGCTCGCGGCCGCCCCGGGGATCCTCTAGAGGC
CCTTATGGCCttagaagtatacggttttgttgaac-3′
(capital letters: adaptor
sequence)

Each PCR reaction solution used herein contained primer set M1 or M2 (a 15-pM primer each), 3 μl of cDNA, 3 μl of 10×PCR buffer, 3 μl of dNTPs, 0.3 μl of polymerase enzyme, and 20.4 μl of sterile water, in a total volume of 30 μl (10×PCR buffer, dNTPs, and polymerase enzyme used were provided in the TOYOBO rTaq DNA Polymerase; Code No. TAP-211). The PCR was performed with heat denaturation at 95° C. for 5 minutes, followed by 40 cycles of denaturation at 94° C. for 0.5 minutes, annealing at 61° C. for 0.5 minutes, and elongation at 72° C. for 1 minute.

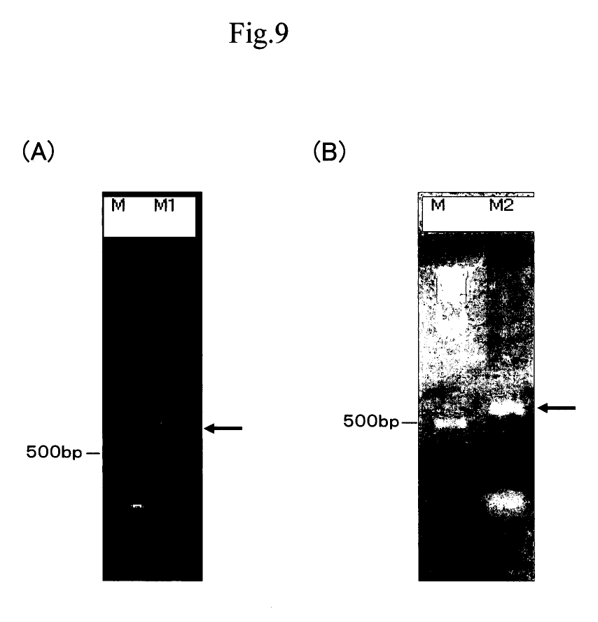

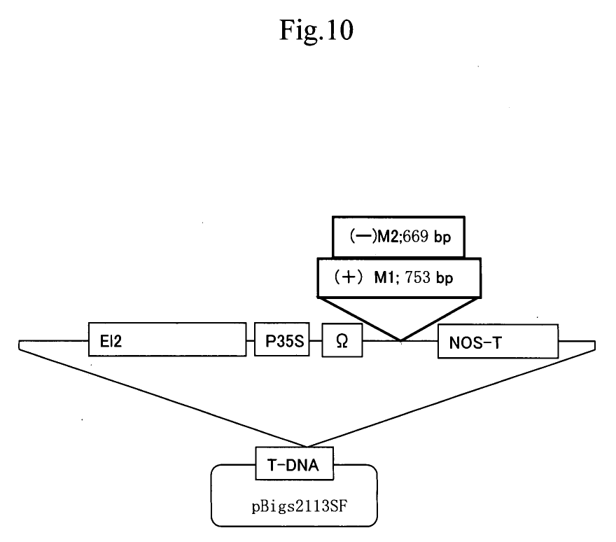

The thus obtained PCR products were electrophoresed, so as to confirm the sizes of the amplified products. As shown in FIG. 9, bands corresponding to the above amplification sizes were confirmed for primer sets M1 and M2, respectively. Subsequently, PCR-amplified fragments corresponding to these bands were excised and then purified by a standard method. After purification, these PCR fragments were each inserted into an expression vector pBigs2113SF (constructed by adding two Sfi I sites to pBIG2113N; see Taji et al., Plant J29, 417-426) (FIG. 10). The thus obtained recombinant expression vectors were then introduced by electroporation into Escherichia coli competent cells (DH10B strain). Positive colonies were isolated in the presence of 50 mg/L kanamycin. Colony PCR was performed using each isolated colony as a template and primer set M1 or M2. PCR products were then electrophoresed. For each of primer sets M1 and M2, colonies having expression vectors into which inserts of a size identical to the relevant amplification size had been inserted were confirmed.

Colonies into which the inserts of the target size had been inserted were scraped off and then subcultured in media. Subsequently, Escherichia coli plasmids were extracted therefrom by a standard method. The inserts of these plasmids were sequenced. As a result, for both primer sets M1 and M2, the sequence of each insert was confirmed to be identical to the sequence of each amplified fragment predicted from the nucleotide sequence of the miraculin gene.

Agrobacterium strain GV3101 was transformed with each of these Escherichia coli plasmids by electroporation. The transformants were selectively cultured in LB media with 50 mg/L kanamycin.

Subsequently, the miraculin gene was introduced using the thus obtained Agrobacterium transformant into Arabidopsis thaliana by the floral dipping method via Agrobacterium. Specifically, according to the method described in literature (Clough et al., 1995, Plant J. 16, 735-743), the transformed Agrobacterium cells were shake-cultured overnight in LB media with 50 mg/L kanamycin, the bacteria were then harvested by centrifugation and suspended in MS liquid media with 5% sucrose, and subsequently, plant bodies above the ground of Arabidopsis thaliana (wild-type Col-0 (diploid)) whole plants at about 1.5 months after seeding were immersed in the suspensions so that the plant bodies were infected with the Agrobacterium. Thereafter, the Arabidopsis thaliana plant bodies were put into bags with zippers. One day after sealing the bags, the bags were opened (unzipped) and the plant bodies were allowed to stand in the bags for 1 day. Next, the plant bodies were removed from the bags and then grown until seeds could be harvested therefrom.

Seeds (Ti) were harvested from the thus treated Arabidopsis thaliana plants and then seeded on BAM media. Drug selection was performed using 20 mg/L hygromycin and 100 mg/L cefotaxime sodium as drugs. As a result, 9 transformants (M1-1, M1-2, M1-3, M1-4, M1-5, M1-6, M1-7, M1-8, and M1-9) were obtained using the products amplified with the use of primer set M1 (hereinafter, referred to as the M1 group). In addition, six transformants (M2-1, M2-2, M2-3, M2-4, M2-5, and M2-6) were obtained using the products amplified with the use of primer set M2 (referred to as the M2 group). These transformants (T1 plants) were transplanted to soil and then grown.

After the plants had been grown until the leaves had grown larger, 2 to 3 leaves (approximately 100 mg) were sampled from each plant individual. Leaf samples derived from the M1 group were combined together and leaf samples derived from the M2 group were also combined together. A taste test was conducted with 6 subjects using the samples as follows. Each subject placed lemon juice in his or her mouth to confirm its sour taste. Subsequently, the subject rinsed his or her mouth, chewed the leaf samples well within his or her mouth for approximately 1 minute, and then expelled the sample. Then the subject tasted lemon juice again. As a result, 6 subjects unanimously confirmed the same change of the taste. For the T1 plants of the M1 group, all of 6 subjects experienced sweet tastes when they had tasted lemon juice after chewing the leaf samples. In contrast, for the T1 plants of the M2 group, none of the 6 subjects experienced a modified taste from the lemon juice that they had tasted after chewing the leaf samples. Therefore, it was confirmed that the plant bodies derived from the M1 group contained large amounts of the miraculin protein in its active form. In contrast, since a modified taste was not confirmed for the plant bodies of the M2 group, it was considered that the plant bodies of the M2 group contained low amounts of the miraculin protein or contained the miraculin protein with weak activity.

Next, seeds were harvested from each individual transformant of the M1 group obtained above. 50 seeds per each of the plant individuals (lines) were seeded on BAM media and then subjected to drug selection with 20 mg/L hygromycin and 100 mg/L cefotaxime sodium. The thus selected transformants (T2 plants) were transplanted at 4 to 9 plant individuals per each of the lines to soil and then grown.

The plants were grown until the leaves grew larger. Subsequently, 2 to 3 leaves (approximately 100 mg) were sampled from each of these T2 plants. The thus sampled leaves were combined together for each line and then modification of taste was confirmed with the. samples. Confirmation was performed in a manner similar to the above, using lemon juice as a sour material.

As a result, for the samples derived from lines M1-4, M1-9, M1-2, and M1-5, the subjects experienced sweet tastes when they had tasted lemon juice after chewing the leaf samples. Particularly for samples derived from line M-4, the subjects experienced strong sweet tastes. Therefore, it was demonstrated that the ability of the plant bodies of the M1 group to express the active miraculin protein was inherited by the following generation from the T1 plants.

Moreover, the protein was extracted by a standard method from the leaf samples of the T2 plants of the M1 group as prepared above (the plant individuals from which the leaf samples had been sampled are listed in the following Table 3) and the T1 plants of the M2 group as prepared above (6 plant individuals of M2-1 to M2-6). The thus extracted protein was subjected to SDS-PAGE under non-reducing conditions, transferred to a membrane, and subjected to the Western blot method using the miraculin-recognizing specific antibody prepared in Example 5 to detect the miraculin protein in each sample.

TABLE 3
Sampling of leaves from the grown T2 plants of the M1 group

| line | M1-1-1 | M1-1-2 | M1-1-3 | M1-1-4 | M1-1-5 | M1-1-6 | M1-1-7 | M1-1-8 |
|---|---|---|---|---|---|---|---|---|
| M1-1 | ∘ | ∘ | ∘ | ∘ | ∘ | ∘ | ∘ | ∘ |
| line | M1-2-1 | M1-2-2 | M1-2-3 | M1-2-4 | M1-2-5 | M1-2-6 | M1-2-7 | M1-2-8 |
| M1-2 | ∘ | ∘ | ∘ | ∘ | ∘ | ∘ | ∘ | ∘ |

| line | M1-3-1 | M1-3-2 | M1-3-3 | M1-3-4 | M1-3-5 | M1-3-6 | M1-3-7 |
|---|---|---|---|---|---|---|---|
| M1-3 | — | ∘ | ∘ | ∘ | — | ∘ | ∘ |

| line | M1-4-1 | M1-4-2 | M1-4-3 | M1-4-4 | M1-4-5 | M1-4-6 | M1-4-7 | M1-4-8 | M1-4-9 |
|---|---|---|---|---|---|---|---|---|---|
| M1-4 | ∘ | ∘ | ∘ | ∘ | ∘ | ∘ | ∘ | ∘ | — |

| line | M1-5-1 | M1-5-2 | M1-5-3 | M1-5-4 | M1-5-5 | M1-5-6 | M1-5-7 | M1-5-8 |
|---|---|---|---|---|---|---|---|---|
| M1-5 | — | ∘ | ∘ | ∘ | — | — | — | — |

| line | | M1-6-1 | | M1-6-2 | | M1-6-3 | | M1-6-4 | |
|---|---|---|---|---|---|---|---|---|---|
| M1-6 | | ∘ | | ∘ | | ∘ | | ∘ | |
| line | M1-7-1 | M1-7-2 | M1-7-3 | M1-7-4 | M1-7-5 | M1-7-6 | M1-7-7 | M1-7-8 | M1-7-9 |
| M1-7 | ∘ | ∘ | ∘ | ∘ | ∘ | ∘ | ∘ | ∘ | ∘ |

| line | M1-7-1 | M1-7-2 | M1-7-3 | M1-7-4 | M1-7-5 | M1-7-6 | M1-7-7 |
|---|---|---|---|---|---|---|---|
| M1-9 | ∘ | ∘ | ∘ | ∘ | ∘ | ∘ | ∘ |

*The upper column for each line indicates the name of each individual T2 plant.
“∘” indicates a plant individual from which leaves were sampled for Western blot analysis.

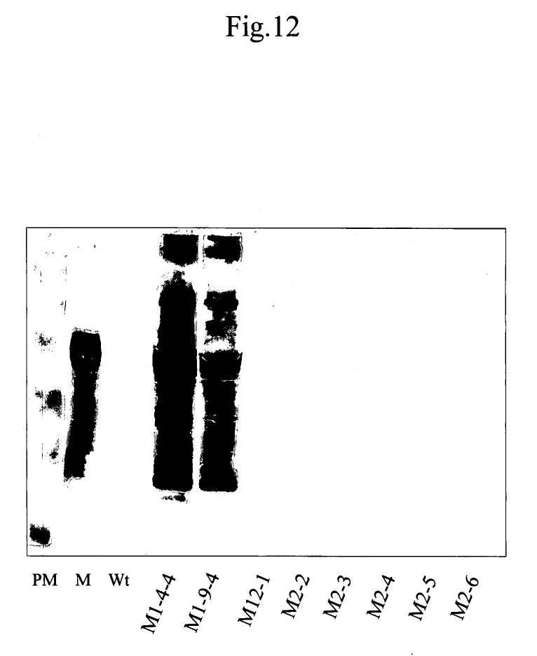

FIG. 11 and FIG. 12 show such representive results. As shown in FIG. 11, for the M1 group, bands with the same mobility as that of the purified miraculin protein (dimer) were observed for the T2 plants of all lines examined.

In the meantime, as shown in FIG. 12, bands with the same mobility as that of the purified miraculin protein (dimer) were observed for plant individuals M1-4-4 and M1-9-4 of the M1 group. However, for the plant individuals of the M2 group, bands observed with the same mobility as that of the purified miraculin protein (dimer) were all very faint.

The results of the above taste test and Western blot demonstrated that the miraculin protein was expressed and existed in an active dimeric form in the transgenic Arabidopsis thaliana plants into which DNA encoding the miraculin protein containing a signal-like peptide sequence had been introduced (M1 group). In contrast to miracle fruit typically having a weight of approximately 1.5 g per fruit, the expression and the activity of miraculin protein could be confirmed in only approximately 100 mg of Arabidopsis thaliana leaves. Furthermore, it was demonstrated that the miraculin protein was produced in only small amounts in transgenic Arabidopsis thaliana plants into which DNA encoding the miraculin protein portion and not containing the signal-like peptide sequence had been introduced (M2 group). Hence, it was concluded that a nucleotide sequence encoding a signal-like peptide sequence is required for expression of the miraculin gene.

### INDUSTRIAL APPLICABILITY

The use of recombinant plants into which the miraculin gene has been introduced, as provided according to the present invention, enables a stable supply of a large amount of miraculin. According to the present invention, miraculin is expressed in general agricultural food crops, which enables miraculin-expressing plants to be used as daily foodstuffs. Furthermore, miraculin can be expressed in such crops at the same level as in miracle fruit. Thus, the expressed miraculin is extracted and then can also be used as a food additive. The use of recombinant plants into which the miraculin gene has been introduced and the plant materials thereof facilitates the use of miraculin for diet or medical purposes.

All publications, patents, and patent applications cited herein are incorporated herein by reference in their entirety.

1. A transgenic plant having taste-modifying activity, into which a miraculin gene is introduced.

2. The transgenic plant according to claim 1, wherein the miraculin gene is at least one of the following miraculin genes (a) to (f):
(a) a gene comprising the nucleotide sequence as shown in SEQ ID NO: 1;
(b) a gene comprising a DNA that hybridizes under stringent conditions to the DNA comprising the nucleotide sequence as shown in SEQ ID NO: 1 and encodes a protein having taste-modifying activity;
(c) a gene encoding a protein that comprises the amino acid sequence as shown in SEQ ID NO: 2;
(d) a gene encoding a protein that comprises an amino acid sequence derived from the amino acid sequence as shown in SEQ ID NO: 2 by deletion, substitution, or addition of one or several amino acids and has taste-modifying activity;
(e) a gene comprising a nucleotide sequence that has 70% or more identity with the nucleotide sequence as shown in SEQ ID NO: 1 and encoding a protein that has taste-modifying activity; and
(f) a gene encoding a protein that comprises an amino acid sequence having 85% or more identity with the amino acid sequence as shown in SEQ ID NO: 2 and has taste-modifying activity.

3. The transgenic plant according to claim 1, wherein said taste-modifying activity is an activity that converts sourness to sweetness.

4. The transgenic plant according to claim 1, wherein said plant is lettuce, tomato, strawberry, or Arabidopsis thaliana.

5. The transgenic plant according to claim 1, wherein said plant is a diploid plant.

6. A method for producing miraculin, comprising extracting miraculin from the transgenic plant according to claim 1.

7. A food additive, comprising miraculin produced in the transgenic plant according to claim 1.

8. A food or drink, comprising miraculin produced in the transgenic plant according to claim 1.

9. The food or drink according to claim 8, which is used for a patient with diabetes.

10. An antidiabetic drug, comprising miraculin produced in the transgenic plant according to claim 1.

11. The transgenic plant according to claim 2, wherein said taste-modifying activity is an activity that converts sourness to sweetness.

12. The transgenic plant according to claim 2, wherein said plant is lettuce, tomato, strawberry, or Arabidopsis thaliana.

13. The transgenic plant according to claim 3, wherein said plant is lettuce, tomato, strawberry, or Arabidopsis thaliana.

14. The transgenic plant according to claim 2, wherein said plant is a diploid plant.

15. The transgenic plant according to claim 3, wherein said plant is a diploid plant.

16. The transgenic plant according to claim 4, wherein said plant is a diploid plant.

17. A method for producing miraculin, comprising extracting miraculin from the transgenic plant according to claim 2.

18. A method for producing miraculin, comprising extracting miraculin from the transgenic plant according to claim 3.

19. A method for producing miraculin, comprising extracting miraculin from the transgenic plant according to claim 4.

20. A method for producing miraculin, comprising extracting miraculin from the transgenic plant according to claim 5.
